(19) United States
(12) Patent Application Publication
(10) Pub. No.: US 2024/0374281 A1
(43) Pub. Date: Nov. 14, 2024

(54) PUNCTURE NEEDLE WITH SURFACE STRUCTURE CAPABLE OF ENHANCING ULTRASONIC REFLECTION

(71) Applicants: Shanghai Ruili Medical Instrument Co., Ltd., Shanghai (CN); Shanghai Liwei Medical Technology Development Co., Ltd., Shanghai (CN)

(72) Inventors: Fengcang MA, Shanghai (CN); Guosen SHAO, Shanghai (CN)

(73) Assignees: Shanghai Ruili Medical Instrument Co., Ltd., Shanghai (CN); Shanghai Liwei Medical Technology Development Co., Ltd., Shanghai (CN)

(21) Appl. No.: 18/196,812

(22) Filed: May 12, 2023

(51) Int. Cl. A61B 17/34

(52) U.S. Cl. CPC A61B 17/3403; A61B 17/3401; A61B 2017/00526; A61B 2017/3413

(57) ABSTRACT

Disclosed is a puncture needle with a surface structure capable of enhancing ultrasonic reflection. The puncture needle is a tubular structure with an inner channel, an ultrasonic reflection enhancement lattice is arranged on a peripheral surface of a needle body near a needle tip. The ultrasonic reflection enhancement lattice is formed by uniformly arranging multiple triangular pyramidal pits, front side surfaces of the triangular pyramidal pits form an ultrasonic reflection mirror, and a front included angle between the ultrasonic reflection mirror and the central axis of the puncture needle is from 30 degrees to 60 degrees. A tungsten-containing alloy layer is prepared on a surface of the needle body of the puncture needle. The ability of the puncture needle in ultrasonic positioning can be improved by the disclosed surface structure and the tungsten-containing coating.

### TECHNICAL FIELD

The present disclosure relates generally to the technical field of medical devices, and in particular to a puncture needle with a surface structure capable of enhancing ultrasonic reflection.

### BACKGROUND

Puncture needle is mainly used for nerve block surgery for anesthetic purposes. In conventional nerve block, blind exploration is mainly based on the anatomical landmarks of peripheral nerves, and the target nerve is determined based on sensation, followed by effective puncture. The conventional nerve block is often affected by obesity, individual anatomical differences, trauma and anatomical variation, which leads to unclear anatomical marks, inability to accurately locate nerve block sites, inability to inject anesthetics into the ideal nerve block site, and low success rate of block or unsatisfactory anesthetic effect. To solve this problem, methods such as increasing the dosage of anesthetics or extending the scope of anesthesia are often adopted clinically to achieve the effect of nerve block, but adverse events such as adverse reactions of anesthetics and injury to blood vessels and nerves often occur in use. In another nerve block surgery, puncture positioning is achieved by observing the response of responsible muscles after the target nerve is subjected to electrical stimulation with the help of electrical stimulator. However, this surgical method has complicated positioning operation process and requires many attempts, which may cause great discomfort to patients.

In recent years, with the continuous development and progress of ultrasound technology, ultrasound guidance can achieve visually positioning to display specific anatomical structures of the site to be anesthetized through images, accurately guide the specific direction and depth of anesthesia puncture, achieve accurate anesthesia, reduce the dosage of anesthetics and reduce the incidence of adverse anesthesia events, so ultrasound guidance is gradually widely used in nerve block. Image-guided surgery is an important progress in the medical field, the spatial position information of a surgical device can be localized in real time during surgery to present three-dimensional spatial structure information of the surgical device in the lesion area for doctors, thus enabling doctors to operate accurately and effectively.

The principle of ultrasound imaging (US) is that echoes such as reflection and scattering generated by ultrasonic waves passing through human organs are received by ultrasonic probes for imaging and display. Ultrasound imaging has the advantages of simple operation, real-time imaging, low price and no radiation, and has been widely used in medical diagnosis. In ultrasound image-guided surgery, surgical device calibration is an indispensable step in image-guided surgery, the spatial position information can only be known after calibration, and then the subsequent related work can be completed. The calibration accuracy of surgical devices directly affects the overall accuracy of the whole surgery. In order to avoid obvious injury to patients caused by the surgery, the diameter of puncture needle is as small as possible, which makes a detectable ultrasonic reflection area limited. Moreover, due to the needs of surgical operation and the individual situation of patients, the puncture angle may be quite different. Therefore, the ultrasonic development at the needle tip of the puncture needle is difficult. Visualization of the tip of the puncture needle is an important but difficult technical problem in ultrasound-guided peripheral nerve block operation, and the visualization of needle tip is an urgent problem to be solved in ultrasound-guided nerve block at present.

An existing common puncture needle is a tubular structure with a smooth surface, when this puncture needle is ultrasonically developed and when the puncture needle is at a certain angle with an ultrasonic plane, the reflected wave and the incident wave are not in the same direction due to the characteristics of ultrasonic mirror reflection (a reflection angle is equal to an incident angle), and an ultrasonic receiver cannot receive ultrasonic signals reflected by a puncture needle mirror, as shown in FIG. 1. In this case, all the reflected waves of ultrasound imaging come from diffuse reflection on the surface of the puncture needle, and the low intensity of reflected waves leads to poor ultrasonic development effect of the puncture needle, which makes doctors difficult to determine the position of the needle tip of the puncture needle, and increases the difficulty and risk of surgery.

An ultrasonic developing catheter and a modeling process are disclosed in the invention with Application No. CN201310540161, belonging to the technical field of medical devices. Reflection concave holes are processed on a soft tube body to concentrate and reflect ultrasound, and thus the soft tube can be clearly localized and tracked by using ultrasonic developing equipment.

An ultrasonic developing catheter is disclosed in the invention with Application No. CN201510110547. The ultrasonic developing catheter includes a hollow tube body, the tube body is a medical soft catheter, high-molecular material fiber yarns are arranged in the wall of the tube body, and the high-molecular material fiber yarns are mutually crossed and overlapped to form a netted woven mesh layer, so that ultrasonic developing areas of different densities are formed between the tube body and the woven mesh layer, the ultrasonic developing capacity is improved, and the developing at the tip of the catheter is facilitated.

An electromyography-guided injection needle capable of enhancing ultrasonic development is disclosed in the invention with Application No. CN201420754040. A structure capable of enhancing ultrasonic development is arranged at one end, close to a needle tip, of a needle tube, which is at least one concave-convex structure regularly distributed on an outer surface of the needle tube and a smooth micro-toothed structure located on an inclined plane of the needle tip. The ultrasonic reflection area of the tip of the injection needle is increased through the concave-convex structure, and the developing property is increased by increasing the ultrasonic reflection wave intensity, and thus the positioning accuracy of muscles is improved.

A puncture needle with enhanced ultrasonic development/double-lumen oocyte retrieval needle is disclosed in the invention with Application No. CN201821551080/CN201821551069. In accordance with above application, an ultrasonic development enhancement belt set is arranged on an outer wall face of a needle tube, the ultrasonic development enhancement belt is composed of linear grooves arranged in an axial direction of the needle tube, and the groove line type is a curve with periodic changes, so that the ultrasound imaging of the puncture needle shows a light and dark scale, which is easy for an operator to observe in real time directly on a display of an ultrasound imaging system during surgery.

Even if the surface of the existing puncture needle is designed with a reflection lattice, the reflection surface in the reflection lattice is not designed according to an included angle between a puncture direction of the puncture needle and an ultrasonic surface during use, so the reflection lattice on the surface only increases the area of ultrasonic reflection, and the maximum development effect cannot be achieved by ultrasonic mirror reflection.

### SUMMARY

An objective of the present disclosure is to provide a puncture needle with a surface structure capable of enhancing ultrasonic reflection. The ability of the puncture needle in ultrasonic development can be improved by changing the surface structure of the puncture needle.

In accordance with a puncture needle with a surface structure capable of enhancing ultrasonic reflection of the present disclosure, the puncture needle is a tubular structure with an inner channel, and an ultrasonic reflection enhancement lattice is arranged on a peripheral surface of a needle body near a needle tip. The ultrasonic reflection enhancement lattice is formed by uniformly arranging multiple triangular pyramidal pits, front side surfaces of the triangular pyramidal pits form an ultrasonic reflection mirror, a front included angle between the ultrasonic reflection mirror and the central axis of the puncture needle is from 30 degrees to 60 degrees. A tungsten-containing alloy layer is prepared on a surface of the needle body of the puncture needle.

During the use of the puncture needle in surgery, when an included angle between a direction of the puncture needle and an ultrasonic detection surface (ultrasonic generating/receiving surface) is equal to or close to the front included angle between the ultrasonic reflection mirror and the central axis of the puncture needle, reflected waves have a direction basically the same as that of incident waves. The reflection mirror in the ultrasonic reflection enhancement lattice designed by the present disclosure can obtain the maximum ultrasonic development enhancement effect.

In order to obtain a stable ultrasonic development effect, the inventor has carried out a large number of theoretical calculations and experimental verifications on design parameters of the ultrasonic reflection enhancement structure and obtained preferred design parameters: an outer diameter of the puncture needle is from 0.3 mm to 2.0 mm, and the number of pits per circumference is four to twelve, the number of pits per circumference increases gradually with the increase of the outer diameter of the puncture needle, an interval between adjacent pits in a circumferential direction is from 0.1 mm to 0.4 mm, and a side length of the ultrasonic reflection mirror is from 0.07 mm to 0.25 mm. Further, a wall thickness of the puncture needle is ⅙ to ¼ of the outer diameter, and a distance from an apex of the triangular pyramidal pit to the outer wall in a diametrical direction is ½ of the wall thickness.

In order to further increase the ultrasonic reflection effect on the surface of the puncture needle, in-depth research is also conducted from the angle of material selection. Ultrasonic reflection may occur when ultrasonic waves are transmitted from human tissues with low acoustic impedance to a tissue/metal interface, the greater the impedance of a metal material, the stronger the reflection effect. Taking a medical puncture needle as an example, the medical puncture needle is widely made of stainless steel, with acoustic impedance of about 30 times that of human soft tissues. Ultrasonic waves in the human tissues may generate remarkable reflection effect when encountering a stainless-steel surface. The acoustic impedance of nickel is about 35 times that of human soft tissues, the acoustic impedance of platinum is about 45 times that of human soft tissues, and the acoustic impedance of tungsten is about 70 times that of human soft tissues. Therefore, in terms of ultrasonic reflection capacity of materials, the ultrasonic reflection capacity of the following materials is as follows: tungsten >platinum>nickel>stainless steel. Therefore, the technical route of a coating with high tungsten content is used to improve the ultrasonic reflection capacity.

According to the present disclosure, a tungsten-containing alloy layer is prepared by using an electroplating method on the surface of the puncture needle with the ultrasonic reflection enhancement lattice. Based on the tungsten content in the coating and the surface quality of the coating, a plating solution formula and electroplating process with high tungsten content and excellent surface quality are preferred.

Specifically, the alloy layer is made of tungsten-nickel alloy, and has an alloy layer thickness from 2 μm to 20 μm.

Specifically, the tungsten-containing alloy layer is prepared by using an electroplating method, and electroplating solution is prepared from sodium tungstate (Na₂WO₄) with a concentration of 18.0 g/L to 54.0 g/L and nickel sulfate (NiSO₄) with a concentration of 2.0 g/L to 6.0 g/L; a concentration ratio of the sodium tungstate to the nickel sulfate is from 7.0 to 9.0, and a pH value of the electroplating solution is from 4.0 to 7.0. During electroplating, a current density on the surface of the puncture needle is controlled at 3.0 A/dm² to 18.0 A/dm², and the temperature is controlled at 20° C. to 70° C.

Compared with the existing puncture needle ultrasonic development technology, a needle tip of the puncture needle adopted by the present disclosure is provided with an enhancement lattice of ultrasonic reflection mirror, which increases the positioning accuracy of ultrasonic development and can significantly reduce the surgical risk. In the existing common puncture needle with a smooth surface, when the puncture needle and an ultrasonic plane have a certain included angle (especially when the angle is large, such as more than 20 degrees), the directions of the reflected waves and incident waves are not in the same orientation, and an ultrasonic receiver cannot receive an ultrasonic signal reflected by the mirror on the surface of the puncture needle. In this case, the intensity of the reflected waves is usually low, and is prone to leading to poor ultrasonic development effect of the puncture needle, as shown in FIG. 1.

Based on the ultrasound imaging principle and the characteristics of the existing ultrasound imaging equipment, an integrated design of the ultrasonic generator and the receiver and the reflection characteristic that a reflection angle is equal to an incident angle during ultrasonic mirror reflection are adopted. The ability of the puncture needle in ultrasonic development can be improved by changing the surface structure of the puncture needle. An ultrasonic reflection enhancement lattice is processed on the surface of the puncture needle near the needle tip, in which the ultrasonic reflection mirror designed in the reflection enhancement lattice can directly reflect ultrasonic waves to the ultrasonic receiver in an incident direction, thereby avoiding the situation that the reflected waves cannot be detected by the ultrasonic receiver when the puncture needle and the ultrasonic wave incident direction are not at a right angle, and effectively solving the problem of poor development and the like caused by this. Secondly, in order to increase the intensity of ultrasonic reflected waves, the characteristic that the acoustic impedance of tungsten is significantly different from that of human tissue is adopted, a layer of coating with high tungsten content is prepared on the surface of puncture needle, so as to increase the intensity of reflected waves. Compared with the conventional ultrasonic development technology, the ultrasonic reflection enhancement lattice designed by the present disclosure significantly improves the ultrasonic development of the puncture needle, realizes the accurate positioning of the needle tip of the puncture needle during surgery through ultrasonic development, increases the positioning accuracy of ultrasonic development, and can significantly reduce the surgical risk.

### BRIEF DESCRIPTION OF THE DRAWINGS

FIG. 1 is a schematic diagram of an incident direction and a reflection direction of ultrasonic development of a common puncture needle;

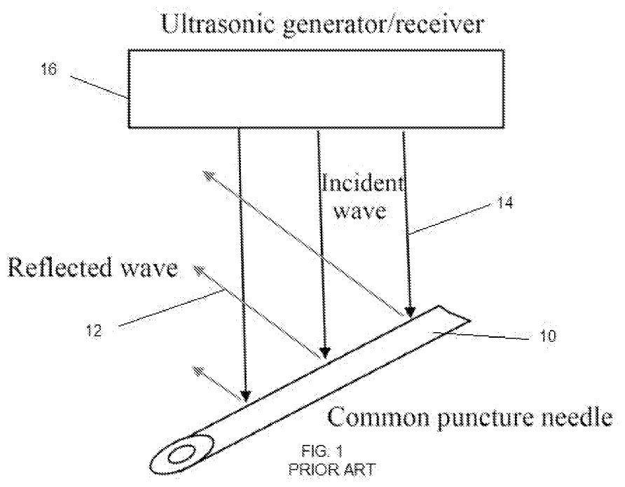

FIG. 2 is a structure schematic diagram of a puncture needle with a surface structure capable of enhancing ultrasonic reflection in accordance with the present disclosure;

FIG. 3 is a schematic diagram of an included angle between an ultrasonic reflection mirror and the central axis of a puncture needle in FIG. 2.

FIG. 4 is a schematic diagram of a puncture angle, incidence and reflection of a puncture needle in accordance with the present disclosure when used.

### DETAILED DESCRIPTION OF THE EMBODIMENTS

The technical solution of the present disclosure is described in detail below by taking a stainless-steel puncture needle for nerve block as an example. The present disclosure is suitable for all ultrasound-guided puncture needles made of metal, especially puncture needles with small diameter and poor ultrasonic development.

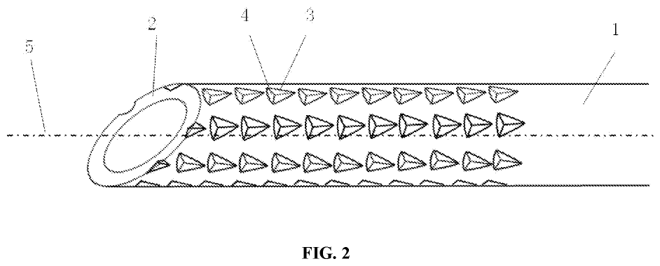

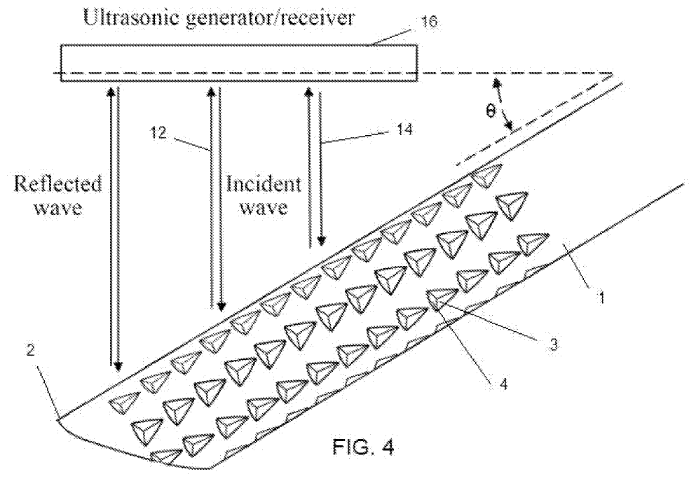

Referring to FIG. 2 through FIG. 4, a puncture needle according to the present disclosure has a needle body 1. The needle body 1 has a tubular structure with an inner channel, and a needle tip 2 at a front end of the needle body 1. An ultrasonic reflection enhancement lattice is arranged on a peripheral surface of the needle body 1 near the needle tip 2, and the ultrasonic reflection enhancement lattice is formed by uniformly arranging multiple triangular pyramidal pits 3. The triangular pyramidal pits 3 are normal triangular pyramids for myopia, and front side surfaces of the triangular pyramidal pits 3 form a smooth ultrasonic reflection mirror 4. A front included angle θ between the ultrasonic reflection mirror 4 and the central axis 5 of the puncture needle is from 30 degrees to 60 degrees. The front included angle θ can be designed to be 30 degrees, 45 degrees and 60 degrees, which can be selected according to the depth of puncture surgery.

The specific processing of the puncture needle according to the present disclosure is as follows.

Step one: A metal tube is processed into a puncture needle tube, and an ultrasonic reflection lattice designed by the present disclosure is processed by a stabbing process near the position of the needle tip, and specific parameters are shown in Table 1.

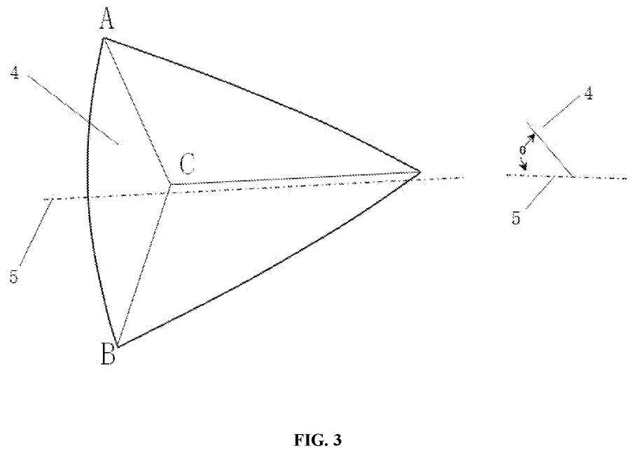

TABLE 1
Design parameters of ultrasonic reflection enhancement
lattice of puncture needle of the present disclosure

| | The number | | | |
|---|---|---|---|---|
| Outer | of | Size of | Wall | Depth of pit |
| diameter | pits per | pit | thickness of | (distance from |
| of puncture | circum- | (AB length, | puncture | point C to outer |
| needle | ference | FIG. 3) | needle | wall, FIG. 3) |
| D/mm | n/pc | L/mm | t/mm | h/mm |
| 0.3-0.5 | 6 | 0.07-0.12 | 0.05-0.1 | 0.025-0.05 |
| 0.5-0.8 | 8 | 0.10-0.16 | 0.1-0.2 | 0.05-0.1 |
| 0.8-1.6 | 10 | 0.12-0.24 | 0.2-0.3 | 0.1-0.2 |
| 1.6-2.0 | 12 | 0.20-0.25 | 0.3-0.4 | 0.15-0.25 |

Step 2: An alloy coating with high tungsten content is prepared on the surface of the puncture needle by electroplating, and a tungsten-nickel alloy coating is obtained by electroplating under different plating solution formulas and technological conditions. Whether there are bubbling, blackening, cracks and other defects on the surface of the coating is observed, and an observation result is used as an evaluation of the surface quality of the puncture needle: good, general and poor. The tungsten content in the coating is measured by EDS (Energy Dispersive Spectroscopy). Based on the surface quality of the coating and the tungsten content in the coating, the suitable electroplating solution formula and electroplating process results are screened and shown in Table 2 (the electroplating time is 5 min).

Step 3: The puncture needle is made by assembling accessories such as a back seat of the puncture needle.

### Example 1

In a case that pH is equal to 7, a current density is 12 A/dm², and the temperature is at 40° C., a puncture needle with an ultrasonic reflection lattice is electroplated, composition ratios of sodium tungstate (Na₂WO₄) to nickel sulfate (NiSO₄) in the used electroplating solution are 18.0:2.0, 25.5:3.0, 36.0:4.0, 45.0:5.0, 54.0:6.0, 59.5:7.0, 68.0:8.0, respectively.

After detection, when the composition ratio of the sodium tungstate (Na₂WO₄) to the nickel sulfate (NiSO₄) in the electroplating solution is from 18.0:2.0 to 54.0:6.0, the tungsten (W) content is from 36.2% to 48.3%, and the surface quality is good. When the composition ratio of the sodium tungstate (Na₂WO₄) to the nickel sulfate (NiSO₄) in the electroplating solution is from 59.5:7.0 to 68.0:8.0, the tungsten (W) content is from 50.4% to 52.1%, and the surface quality is poor.

### Example 2

In a case that pH is equal to 7, a current density is 12 A/dm², and the temperature is at 40° C., a puncture needle with an ultrasonic reflection lattice is electroplated, the mass of nickel sulfate (NiSO₄) in the used electroplating solution remains unchanged and is 5.0 g/L, and concentration ratios of sodium tungstate (Na₂WO₄) to nickel sulfate (NiSO₄) are 7.0, 7.5, 8.0, 8.5, 9.0, 9.5 and 10, respectively.

After detection, when the concentration ratio of the sodium tungstate (Na₂WO₄) to the nickel sulfate (NiSO₄) is from 7.0 to 8.5, the tungsten (W) content is from 32.6% to 46.4%, and the surface quality is good. When the concentration ratio of the sodium tungstate (Na₂WO₄) to the nickel sulfate (NiSO₄) is from 9.5 to 10.0, the tungsten (W) content is from 48.6% to 52.1%, and the surface quality is poor.

### Example 3

In a case that a concentration ratio of sodium tungstate (Na₂WO₄) to nickel sulfate (NiSO₄) in electroplating solution is 9.0, a current density is 12 A/dm², and the temperature is at 40° C., a puncture needle with an ultrasonic reflection lattice is electroplated, and the quality under different pH conditions is observed, and the used pH is 4.0, 5.0, 6.0, 7.0, 8.0 and 9.0, respectively.

After detection, when the pH is from 4.0 to 7.0, the tungsten (W) content is from 38.0% to 43.4%, and the surface quality is good. When the pH is 8.0, the tungsten (W) content is 39.4%, and the surface quality is average. When the pH is 9.0, the tungsten (W) content is 37.2%, and the surface quality is poor.

### Example 4

In a case that a concentration ratio of sodium tungstate (Na₂WO₄) to nickel sulfate (NiSO₄) in electroplating solution is 9.0, pH is 7.0, and the temperature is at 40° C., a puncture needle with an ultrasonic reflection lattice is electroplated, and the quality under different current densities is observed, the used current densities are 3.0, 6.0, 9.0, 12.0, 15.0, 18.0 and 21.0, respectively.

After detection, when the current density is from 3.0 A/dm² to 18.0 A/dm², the tungsten (W) content is from 33.3% to 49.0%, and the surface quality is good. When the current density is 21.0 A/dm², the tungsten (W) content is 50.4%, and the surface quality is poor.

### Example 5

In a case that a concentration ratio of sodium tungstate (Na₂WO₄) to nickel sulfate (NiSO₄) in electroplating solution is 9.0, pH is 7.0, and a current density is 12 A/dm², a puncture needle with an ultrasonic reflection lattice is electroplated, and the quality under different temperature conditions is observed, and the used temperatures are 10° C., 20° C., 50° C., 70° C. and 80° C., respectively.

After detection, when the temperature is at 20° C. to 70° C., the tungsten (W) content is from 41.63% to 46.1%, and the surface quality is good. When the temperature is at 10° C. and 80° C., the tungsten (W) content is 36.3% and 48.3%, and the surface quality is poor.

In conclusion, the optimal electroplating solution formula and electroplating process are as follows: the sodium tungstate (Na₂WO₄) has a concentration of 18.0 g/L to 54.0 g/L, and the nickel sulfate (NiSO₄) has a concentration of 2.0 g/L to 6.0 g/L, the concentration ratio of the sodium tungstate to the nickel sulfate is from 7.0 to 9.0, a pH value is from 4.0 to 7.0, the current density is controlled at 3.0 A/dm² to 18.0 A/dm², and the temperature is controlled at 20° C. to 70° C.

TABLE 2
Tungsten content and surface quality of the coating under different electroplating
solution formulas and electroplating process parameters

| | | | Concen- | | Current | | W | |
|---|---|---|---|---|---|---|---|---|
| Serial | Na₂WO₄ | NiSO₄ | tration | pH | density | Tempera- | content | Surface |
| number | (g/L) | (g/L) | ratio | value | (A/dm²) | ture (° C.) | (%) | quality |
| 1 | 18.0 | 2.0 | 9.0 | 7.0 | 12.0 | 40 | 36.2 | Good |
| 2 | 25.5 | 3.0 | 8.5 | 7.0 | 12.0 | 40 | 40.6 | Good |
| 3 | 36.0 | 4.0 | 9.0 | 7.0 | 12.0 | 40 | 43.1 | Good |
| 4 | 45.0 | 5.0 | 9.0 | 7.0 | 12.0 | 40 | 45.9 | Good |
| 5 | 54.0 | 6.0 | 9.0 | 7.0 | 12.0 | 40 | 48.3 | Good |
| 6 | 59.5 | 7.0 | 8.5 | 7.0 | 12.0 | 40 | 50.4 | Poor |
| 7 | 68.0 | 8.0 | 8.5 | 7.0 | 12.0 | 40 | 52.1 | Poor |
| 8 | 35.0 | 5.0 | 7.0 | 7.0 | 12.0 | 40 | 32.6 | Good |
| 9 | 37.5 | 5.0 | 7.5 | 7.0 | 12.0 | 40 | 34.7 | Good |
| 10 | 40.0 | 5.0 | 8.0 | 7.0 | 12.0 | 40 | 38.7 | Good |
| 11 | 42.5 | 5.0 | 8.5 | 7.0 | 12.0 | 40 | 43.6 | Good |
| 12 | 45.0 | 5.0 | 9.0 | 7.0 | 12.0 | 40 | 46.4 | Good |
| 13 | 47.5 | 5.0 | 9.5 | 7.0 | 12.0 | 40 | 48.6 | Poor |
| 14 | 50.0 | 5.0 | 10.0 | 7.0 | 12.0 | 40 | 52.1 | Poor |
| 15 | 36.0 | 4.0 | 9.0 | 4.0 | 12.0 | 40 | 38.0 | Good |
| 16 | 36.0 | 4.0 | 9.0 | 5.0 | 12.0 | 40 | 43.1 | Good |
| 17 | 36.0 | 4.0 | 9.0 | 6.0 | 12.0 | 40 | 45.6 | Good |
| 18 | 36.0 | 4.0 | 9.0 | 7.0 | 12.0 | 40 | 43.4 | Good |
| 19 | 36.0 | 4.0 | 9.0 | 8.0 | 12.0 | 40 | 39.4 | General |
| 20 | 36.0 | 4.0 | 9.0 | 9.0 | 12.0 | 40 | 37.2 | Poor |
| 21 | 42.5 | 5.0 | 8.5 | 7.0 | 3.0 | 40 | 33.3 | Good |
| 22 | 42.5 | 5.0 | 8.5 | 7.0 | 6.0 | 40 | 37.4 | Good |
| 23 | 42.5 | 5.0 | 8.5 | 7.0 | 9.0 | 40 | 41.9 | Good |
| 24 | 42.5 | 5.0 | 8.5 | 7.0 | 12.0 | 40 | 43.7 | Good |
| 25 | 42.5 | 5.0 | 8.5 | 7.0 | 15.0 | 40 | 47.1 | Good |
| 26 | 42.5 | 5.0 | 8.5 | 7.0 | 18.0 | 40 | 49.0 | Good |
| 27 | 42.5 | 5.0 | 8.5 | 7.0 | 21.0 | 40 | 50.4 | Poor |
| 28 | 42.5 | 5.0 | 8.5 | 7.0 | 12.0 | 10 | 36.3 | Poor |
| 29 | 42.5 | 5.0 | 8.5 | 7.0 | 12.0 | 20 | 41.6 | Good |
| 30 | 42.5 | 5.0 | 8.5 | 7.0 | 12.0 | 50 | 44.3 | Good |
| 31 | 42.5 | 5.0 | 8.5 | 7.0 | 12.0 | 70 | 46.1 | Good |
| 32 | 42.5 | 5.0 | 8.5 | 7.0 | 12.0 | 80 | 48.3 | Poor |

Finally, it should be noted that the above embodiments are only used to illustrate the technical scheme of the present disclosure rather than limiting. Although the present disclosure has been described in detail with reference to the foregoing embodiments, it should be appreciated by those of ordinary skill in the art that the technical solutions described in the foregoing embodiments can still be modified or some technical features thereof can be equivalently replaced. These modifications or replacements do not make the essence of the corresponding technical solutions from the spirit and scope of the technical solutions of the various embodiments of the present disclosure.

What is claimed is:

1. A puncture needle with a surface structure capable of enhancing ultrasonic reflection, wherein the puncture needle is a tubular structure with an inner channel, an ultrasonic reflection enhancement lattice is arranged on a peripheral surface of a needle body near a needle tip, the ultrasonic reflection enhancement lattice is formed by uniformly arranging a plurality of triangular pyramidal pits, front side surfaces of the triangular pyramidal pits form an ultrasonic reflection mirror, a front included angle between the ultrasonic reflection mirror and the central axis of the puncture needle is from 30 degrees to 60 degrees, and a tungsten-containing alloy layer is prepared on a surface of the needle body of the puncture needle.

2. The puncture needle according to claim 1, wherein the alloy layer is a tungsten-nickel alloy and has a thickness from 2 μm to 20 μm.

3. The puncture needle according to claim 2, wherein the tungsten-containing alloy layer is prepared by using an electroplating method, and electroplating solution is prepared from sodium tungstate (Na₂WO₄) with a concentration of 18.0 g/L to 54.0 g/L and nickel sulfate (NiSO₄) with a concentration of 2.0 g/L to 6.0 g/L; a concentration ratio of the sodium tungstate to the nickel sulfate is from 7.0 to 9.0, and a pH value of the electroplating solution is from 4.0 to 7.0.

4. The puncture needle according to claim 3, wherein during electroplating, a current density on a surface of the puncture needle is controlled at 3.0 A/dm² to 18.0 A/dm², and the temperature is controlled at 20° C. to 70° C.
